(19) United States
(12) Patent Application Publication
(10) Pub. No.: US 2023/0323263 A1
(43) Pub. Date: Oct. 12, 2023

(54) SPECIAL CULTURE APPARATUS FOR 3D BIOLOGICAL TISSUE, AND METHOD FOR PREPARING BLOCK-SHAPED CULTURED MEAT

(71) Applicant: CHINA MEAT RESEARCH CENTER, Beijing (CN)

(72) Inventors: Shouwei WANG, Beijing (CN); Shilei LI, Beijing (CN); Yingying LI, Beijing (CN); Wenting LIU, Beijing (CN); Yushuang LI, Beijing (CN)

(21) Appl. No.: 18/022,872

(22) PCT Filed: Nov. 27, 2020
(86) PCT No.: PCT/CN2020/132217

§ 371 Date: Feb. 23, 2023

(30) Foreign Application Priority Data
Sep. 29, 2020 (CN) 202011055204.7

(51) Int. Cl. C12M 3/00; C12M 1/00; C12N 5/077

(52) U.S. Cl. CPC C12M 21/08; C12M 29/10; C12M 29/18; C12N 5/0659; C12N 2501/11; C12N 2513/00; C12N 2501/33; C12N 2533/78

(57) ABSTRACT

The present disclosure provides a special culture apparatus for a 3D biological tissue, and a method for preparing block-shaped cultured meat. The special culture apparatus for a 3D biological tissue includes: a 3D biological tissue culture tank for accommodating a 3D biological tissue, and a liquid storage tank for containing a culture medium; the 3D biological tissue culture tank is connected to the liquid storage tank by means of a pipeline to form a circuit for the culture medium to circularly flow; and an opening of the 3D biological tissue culture tank is provided with a sealing plug, an inner side of the sealing plug is provided with a plurality of culture medium infusion needles that penetrate the 3D biological tissue when in use.

### CROSS REFERENCE TO RELATED APPLICATION

This patent application claims the benefit and priority of Chinese Patent Application No. 202011055204.7 filed with the China National Intellectual Property Administration on Sep. 29, 2020, and entitled “SPECIAL CULTURE APPARATUS FOR 3D BIOLOGICAL TISSUE, AND METHOD FOR PREPARING BLOCK-SHAPED CULTURED MEAT”, the disclosure of which is incorporated by reference herein in its entirety as part of the present application.

### TECHNICAL FIELD

The present disclosure relates to the technical field of cultured meat preparation, in particular to a special culture apparatus for a 3D biological tissue and a method for preparing block-shaped cultured meat.

### BACKGROUND

Cultured meat, also called lab-grown meat, clean meat, and in vitro meat, is a kind of cell-based artificial meat. This kind of meat can sustainably supply real animal proteins for humans because it can bypass animal feeding. It is the most likely solution for human in the future to the dilemma of meat production and consumption. (Wang S W et al. Classification of artificial meat and suggestions on normalization of nomenclature for related terms. Food Science, 2020, 41(11): 310-316).

At present, the preparation technology of cultured meat is still in the developing phase, and a plurality of key technologies such as cell source required for preparation, cell culture in vitro, and 3D in vitro cell shaping still need to be further broken through or improved (See Dong G L. “Progress in research on cultured meat and relevant patent applications”. China Invention and Patent, 2019, 16(7): 71-75)), among which, the 3D in vitro cell shaping and culture are closely related to the eating taste of the cultured meat. The existing cultured meat preparation technology can only prepare small-sized myotube cell clusters or myotube cells, which are then combined into an edible-scale cultured meat product by using physical methods, for example, adding excipients (Chinese patent application No. CN110730615A), leading to a poor chewing sensation of the cultured meat.

### SUMMARY

Provided in embodiments of the present disclosure is a special culture apparatus for a 3D biological tissue and a method for preparing block-shaped cultured meat. A single large-size block-shaped cultured meat structure unit is unable to be prepared at one time. This defect is overcome by solving the technical problem of 3D culture of animal skeletal muscle satellite cells, so that the control of the 3D configuration and size of the single structure unit thereof can be realized during the preparation of cultured meat.

Provided in embodiments of the present disclosure is a special culture apparatus for a 3D biological tissue, including:
a 3D biological tissue culture tank for accommodating a 3D biological tissue, and
a liquid storage tank for containing a culture medium;
where the 3D biological tissue culture tank is connected to the liquid storage tank by means of a pipeline to form a circuit for the culture medium to circularly flow; and
an opening of the 3D biological tissue culture tank is provided with a sealing plug, with an inner side of the sealing plug provided with a plurality of culture medium infusion needles that penetrate the 3D biological tissue when in use.

Since an ordinary culture apparatus is in a static state in an incubator, cells in the middle part of the 3D biological tissue will not grow normally or will necrose due to insufficient nutrient penetration. The special culture apparatus for a 3D biological tissue provided in the embodiments of the present disclosure allows the middle part of the 3D biological tissue to infiltrate nutrients well through the arrangement of the culture medium infusion needles, and the culture medium is always in a circulating state, which is relatively uniform and can effectively guarantee the uniform growth and differentiation of cells in the 3D biological tissue.

Further provided in embodiments of the present disclosure is a method for preparing block-shaped cultured meat, including steps of:
mixing animal skeletal muscle satellite cells with bioink for 3D bioprinting, and placing a printed 3D animal skeletal muscle satellite cell tissue in the special culture apparatus for a 3D biological tissue provided by the foregoing embodiments of the present disclosure for proliferation, culture and differentiation.

In vitro culture and differentiation in the special culture apparatus for a 3D biological tissue provided by the foregoing embodiments of the present disclosure may effectively guarantee the uniform growth and differentiation of cells in the block-shaped 3D biological tissue to obtain the block-shaped cultured meat.

According to the method for preparing block-shaped cultured meat provided in the embodiments of the present disclosure, the bioink is methacrylic anhydride-modified gelatin (GelMa) and/or nanocellulose.

The GelMa as the bioink for 3D bioprinting of mesenchymal stem cells (MSCs) allows an excellent cell growth and differentiation state, which, together with its excellent culture medium permeability, makes it very suitable for the preparation of the block-shaped cultured meat. In addition, the nanocellulose is well-biocompatible, plant-derived, low in production cost, and edible. The nanocellulose shows excellent biocompatibility as bioink whether used alone or mixed with the GelMa. It is found in experiments that a mixture system of the GelMa and the nanocellulose is more suitable for the preparation of the block-shaped cultured meat than the GelMa alone.

According to the method for preparing block-shaped cultured meat provided in the embodiments of the present disclosure, the animal skeletal muscle satellite cells are porcine skeletal muscle satellite cells or chicken skeletal muscle satellite cells.

Specifically, when the animal skeletal muscle satellite cells are porcine skeletal muscle satellite cells, the animal skeletal muscle satellite cells are mixed with the bioink for 3D bioprinting, a printed 3D animal skeletal muscle satellite cell tissue is placed in the special culture apparatus for a 3D biological tissue provided in the embodiments of the present disclosure, and the whole is cultured at 36.5-37.5° C. in 5% CO₂; when cell morphology is stable, the proliferation medium is replaced with a differentiation medium until the block-shaped cultured meat is formed.

Herein, the proliferation medium used contains 8-12 ng/mL epidermal growth factor, 0.5-2 ng/mL fibroblast growth factor, 0.005-0.015 mg/L insulin, and 0.3-0.5 μg/mL dexamethasone, and the differentiation medium used contains 0.005-0.015 mg/L insulin.

The foregoing culture medium used allows cells to grow normally under serum-free conditions, and the growth cycle of thereof is consistent with that achieved by a culture medium supplemented with 20% fetal bovine serum (FBS), which is beneficial to reduce the production cost of the cultured meat.

Specifically, when the animal skeletal muscle satellite cells are derived from chicken, the animal skeletal muscle satellite cells are mixed with the bioink for 3D bioprinting, a printed 3D animal skeletal muscle satellite cell tissue is placed in the special culture apparatus for a 3D biological tissue provided in the embodiments of the present disclosure, and the whole is cultured at 40.5-41.5° C. in 5% CO₂; when cell morphology is stable, the proliferation medium is replaced with a differentiation medium until the block-shaped cultured meat is formed.

Herein, the proliferation medium used is a McCoy's 5A Medium supplemented with 10-20% chicken serum or FBS, and the differentiation medium used is a McCoy's 5A Medium supplemented with 0-5% chicken serum or FBS.

The present disclosure finds through experiments that are for the chicken skeletal muscle satellite cells, a conventional culture temperature of 36.5-37.5° C. is not the most suitable, and the effect is better at 40.5-41.5° C. and optimal is at 41° C.

In summary, the use of the special skeletal muscle satellite cell proliferation medium, differentiation medium, and special culture apparatus for a 3D biological tissue provided in the embodiments of the present disclosure is beneficial to efficiently realize the directional differentiation of the skeletal muscle satellite cells from the 3D biological tissue into multinucleated myotube cells. Moreover, for different animal skeletal muscle satellite cells, the special skeletal muscle satellite cell proliferation medium and the differentiation medium are slightly different, which shows specificity thereof.

According to the method for preparing block-shaped cultured meat provided in embodiments of the present disclosure, the animal skeletal muscle satellite cells are obtained by extraction and in vitro culture, where the porcine skeletal muscle satellite cells are extracted from a skeletal muscle tissue of a neonatal animal, and the chicken skeletal muscle satellite cells are extracted from an embryo in an incubator. At this time, the corresponding skeletal muscle satellite cells are most abundant and have higher proliferative activity, which is conducive to subsequent culture.

In some embodiments, the method for extraction is the tissue explant adherent method. The tissue explant adherent method, digestion tissue explant adherent method, and the enzymatic digestion filtration method are commonly used methods for extracting tissue cells. However, for the skeletal muscle satellite cells provided by the present disclosure, the inventors have found that the extraction of the plurality of skeletal muscle satellite cells can be completed in a shorter time by using the tissue explant adherent method, with high extraction efficiency and convenience.

The in vitro culture is implemented by an adherent culture method or a suspension culture method by means of loading on the surface of a microcarrier microsphere.

The present disclosure finds that in addition to conventional growth of the animal skeletal muscle cells in a Petri dish or rapid growth in a cell factory in an adherent manner, the animal skeletal muscle cells may also be loaded on the surface of a microcarrier microsphere to grow rapidly in a suspension culture manner. Both methods may achieve the rapid cell proliferation. Herein, cells obtained by the suspension culture method show greater growth density, and there is a greater possibility to further reduce the production cost and expand the production scale. Specifically, the density of adherent cells is 2×10⁵ cells/cm²; and the density of cells obtained by the suspension culture is 3.5×10⁷ cells/mL (for chickens) or 2.5×10⁷ cells/mL (for pigs).

In some embodiments, when the in vitro culture is performed, a serum-free special medium or a general purpose medium supplemented with 10%-20% FBS may be used, and a preferred medium may be a general purpose medium DMEM supplemented with 20% FBS. The culture is conducted at 36.5-37.5° C. in 5% CO₂.

As a preferred embodiment of the present disclosure, the method for preparing block-shaped cultured meat includes the following steps:
step 1, extraction of animal skeletal muscle satellite cells:
extracting the animal skeletal muscle satellite cells by a tissue explant adherent method;
step 2, in vitro culture of the animal skeletal muscle satellite cells:
culturing extracted animal skeletal muscle satellite cells in vitro by an adherent culture method or a suspension culture method by means of loading on the surface of a microcarrier microsphere, where a culture medium is collective medium DMEM supplemented with 20% FBS, and the culture is conducted at 36.5-37.5° C. in 5% CO₂;
step 3, 3D bioprinting of the animal skeletal muscle satellite cells:
mixing the animal skeletal muscle satellite cells cultured in vitro with bioink for 3D bioprinting, where the bioink is GelMa and/or nanocellulose, and a volume ratio of the bioink is 1%-20%; and
step 4, proliferation culture and differentiation of a 3D animal skeletal muscle satellite cell tissue:
placing a printed 3D animal skeletal muscle satellite cell tissue in the special culture apparatus for a 3D biological tissue provided in the embodiments of the present disclosure, and culturing the whole at 36.5-37.5° C. in 5% CO₂ (for porcine skeletal muscle satellite cells)/40.5-41.5° C. in 5% CO₂ (for chicken skeletal muscle satellite cells); after 1-2 days, with stable cell morphology, replacing a proliferation medium with a differentiation medium; and after 3-5 days, replacing the differentiation medium with the differentiation medium for further culture until the cells differentiate and fuse to form the block-shaped cultured meat.

In the above technical solution, the method for extracting the animal skeletal muscle satellite cells may quickly extract skeletal muscle satellite cells and reduce the damage to the skeletal muscle satellite cells; furthermore, the animal skeletal muscle satellite cells obtained exhibit high purity, uniform and stable morphology, stable sternness maintenance, high regenerative activity, and fast expansion rate.

The suitable media and effective large-scale expansion means adopted in the in vitro culture method of the animal skeletal muscle satellite cells allows for stable, large-scale and efficient expansion of the animal skeletal muscle satellite cells, as well as large cell growth density, low consumption of reagents, and simple operation in the expansion process.

The 3D bioprinting method of the animal skeletal muscle satellite cells can quickly realize the rapid prototyping of cell 3D biological tissues and precise control of the shape. Due to simple operation and great expansion potential, the 3D bioprinting method may be used for large-scale and rapid production and culture of meat structural units.

The proliferation culture and differentiation method of the 3D animal skeletal muscle satellite cell tissue may provide sufficient nutrient supply for in vitro cell 3D biological tissue culture, and ensure that the cells inside the 3D biological tissue are in a good growth state; the method may realize the 3D in situ differentiation of skeletal muscle satellite cells, which may be used for the rapid preparation of structural units of block-shaped cultured meat and is conducive to forming the chewing sensation of cultured meat products.

Further provided in embodiments of the present disclosure is block-shaped cultured meat, where the block-shaped cultured meat is prepared by the method for preparing block-shaped cultured meat according to any of the foregoing technical solutions

The special culture apparatus for a 3D biological tissue provided in the embodiments of the present disclosure may effectively guarantee the uniform growth and differentiation of cells in the 3D biological tissue and is suitable for the preparation of block-shaped cultured meat. The method for preparing block-shaped cultured meat provided in the embodiments of the present disclosure can significantly increase the preparation scale of cultured animal meat, and the size of the prepared culture meat reaches 1×1×1 cm³. Compared with the prior art, namely small-sized myotube cell clusters or myotube cells are first prepared and then bonded together by physical methods, the method for preparing block-shaped cultured meat truly achieves the preparation of high-quality block-shaped cultured meat with a chewing sensation of meat products. The preparation method can be widely used in the fields of artificial meat, cell engineering, regenerative medicine, molecular biology, and the like.

### BRIEF DESCRIPTION OF THE DRAWINGS

FIG. 1 is a structural representation of a special culture apparatus for a 3D biological tissue provided in Example 1 of the present disclosure, where 1 represents a 3D biological tissue culture tank; 2 represents a sealing plug; 3 represents a liquid storage tank; 4 represents a culture medium infusion needle; 5 represents a first differential pressure gauge; 6 represents a filling pump; 7 represents a second differential pressure gauge; and 8 represents a suction pump;

FIGS. 2 and 3 illustrate the morphology of porcine skeletal muscle satellite cells cultured for 48 h and 72 h in step (2) of Example 2 of the present disclosure, respectively;

FIGS. 4 and 5 illustrate the morphology of chicken skeletal muscle satellite cells cultured for 24 h and 48 h in step (2) of Example 3 of the present disclosure, respectively;

FIG. 6 illustrates the morphology of porcine skeletal muscle satellite cells cultured for 72 h in step (2) of Example 4 of the present disclosure;

FIG. 7 illustrates the morphology of chicken skeletal muscle satellite cells cultured for 48 h in step (2) of Example 5 of the present disclosure;

FIG. 8 illustrates the morphology of porcine skeletal muscle satellite cells cultured for 5 days in step (2) of Example 2 of the present disclosure;

FIGS. 9 and 10 illustrate the morphology of a 3D porcine skeletal muscle satellite cell tissue cultured for 10 and 15 days in Example 2 of the present disclosure, respectively;

FIG. 11 illustrates the morphology of a 3D chicken skeletal muscle satellite cell tissue cultured for 15 days in Example 3 of the present disclosure;

FIG. 12 illustrates the myogenesis of porcine muscle stem cells in Example 2 of the present disclosure;

FIG. 13 illustrates the myogenesis of chicken muscle stem cells in Example 3 of the present disclosure;

FIG. 14 illustrates the morphology of porcine skeletal muscle satellite cells extracted by the digestion tissue explant adherent method in Comparative Example 1 of the present disclosure;

FIG. 15 illustrates the morphology of porcine skeletal muscle satellite cells extracted by the tissue explant adherent method in Example 2 of the present disclosure; and

FIG. 16 illustrates the morphology of porcine skeletal muscle satellite cells after static culture in Comparative Example 2 of the present disclosure.

### DETAILED DESCRIPTION OF THE EMBODIMENTS

In order to make the objectives, technical solutions and advantages of the embodiments of the present disclosure clearer, the technical solutions in the embodiments of the present disclosure will be clearly and completely described below with reference to the accompanying drawings in the examples of the present disclosure. Obviously, the described examples are a part of, not all of, embodiments of the present disclosure. All other embodiments obtained by a person of ordinary skill in the art based on the examples of the present invention without creative efforts shall fall within the protection scope of the present invention.

### Example 1

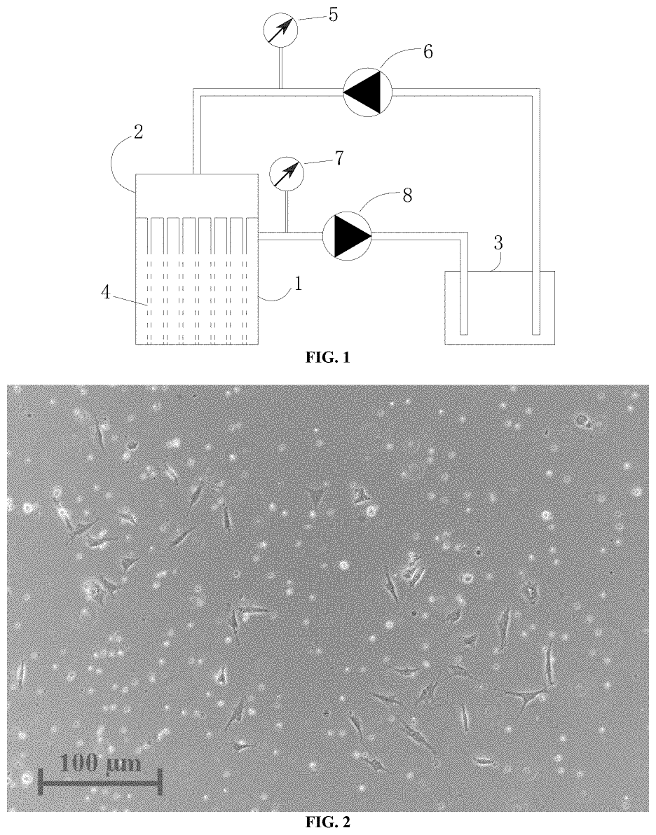

This example of the present disclosure provided a special culture apparatus for a 3D biological tissue, and the structural representation thereof is shown in FIG. 1, including:
a 3D biological tissue culture tank 1 for accommodating a 3D biological tissue, and
a liquid storage tank 3 for containing a culture medium;
where the 3D biological tissue culture tank 1 was connected to the liquid storage tank 3 by means of a pipeline to form a circuit for the culture medium to circularly flow; the pipeline flowing into the 3D biological tissue culture tank 1 was provided with a first differential pressure gauge 5 and a filling pump 6, and a second differential pressure gauge 7 and a suction pump 8 were provided on the pipeline flowing out of the 3D biological tissue culture tank 1; and
an opening of the 3D biological tissue culture tank 1 was provided with a sealing plug 2, with an inner side of the sealing plug 2 provided with a plurality of culture medium infusion needles 4 that penetrate the 3D biological tissue when in use.

### Example 2

This example of the present disclosure provided a method for preparing block-shaped porcine-derived cultured meat, specifically as follows:

(1) Extraction of Porcine Skeletal Muscle Satellite Cells

A newborn pig was sacrificed by CO₂, put in 75% alcohol solution for immersion disinfection for 5 min, and dissected to obtain the skeletal muscle tissue of its thigh; the fascia tissues were torn off, and cut into about 1 mm³ tissue explants with ophthalmic scissors. Phosphate buffered saline (PBS) was added and the tissue explants were washed gently with a pipette. After that, the tissue explants were centrifuged at 700 rpm for 5 min, a resulting supernatant was aspirated, and the lower pellet was the porcine skeletal muscle tissue explant. The above tissue explant was resuspended in DMEM supplemented with 20% FBS, spread on a Petri dish, and placed in an incubator at 37° C. in 5% CO₂ for static culture. After cultured for 3 days, the porcine skeletal muscle tissue explant was pipetted off the Petri dish, and fresh DMEM supplemented with 20% FBS was changed.

(2) Adherent and In Vitro Culture of Porcine Skeletal Muscle Satellite Cells

After the porcine skeletal muscle satellite cells extracted in step (1) covered more than 50% of the bottom area of the Petri dish, the culture medium was discarded, and the cells at the bottom of the Petri dish were rinsed once with Dulbecco's phosphate-buffered saline (DPBS). Thereafter, the cells were digested with 1 mL of 0.25% pancreatin for 4.5 min at 37° C.; then, 1 mL of 10% FBS in PBS was added to terminate the digestion, and the adherent cells were gently pipetted off, transferred to a centrifuge tube, and centrifuged at 900 rpm for 5 min; the supernatant was aspirated, and the lower pellets were primary porcine skeletal muscle satellite cells. Subsequently, the primary porcine skeletal muscle satellite cells were resuspended in 1 mL of DMEM supplemented with 20% FBS and counted. An appropriate quantity of the cells were inoculated in a cell factory (about 3×10⁶ cells/layer), and placed in an incubator at 37° C. in 5% CO₂ for static culture. The skeletal muscle satellite cells were passaged after expansion to cover 80% of the bottom area of the cell factory, and the skeletal muscle satellite cells were continuously expanded.

(3) 3D Bioprinting of Porcine Skeletal Muscle Satellite Cells

Under aseptic conditions, an appropriate quantity of the porcine skeletal muscle satellite cells obtained in step (2) were mixed with a volume of bioink GelMa, so that the volume ratio of the bioink was between 1% and 20%. The porcine skeletal muscle satellite cells were added into a bioprinter for 3D bioprinting into a block or grid in a sterile Petri dish.

(4) Proliferation Culture and Differentiation of 3D Porcine Skeletal Muscle Satellite Cell Tissue

An animal skeletal muscle satellite cell tissue printed in step (3) was placed in the special culture apparatus for a 3D biological tissue provided in Example 1 of the present disclosure, and the whole was cultured at 37° C. in 5% CO₂; after 1-2 days, when the cell morphology was stable, a proliferation medium for porcine skeletal muscle satellite cells (see Table 1 for specific components) was replaced with a differentiation medium for porcine skeletal muscle satellite cells (see Table 2 for specific components). After 3-5 days, the differentiation medium was replaced with the differentiation medium for further culture. Once the skeletal muscle satellite cells inside the 3D biological tissue differentiated and fused to form a unified whole, with the tissue elastic and the tissue surface lustrous, the 3D biological tissue culture and differentiation were completed and a cultured meat structure unit was formed.

TABLE 1
Components of the proliferation medium for
porcine skeletal muscle satellite cells

| | Molecular | Content |
|---|---|---|
| Component name | weight | (mg/L) |
| Glycine | 75 | 30 |
| L-Arginine hydrochloride | 211 | 84 |
| L-Cystine•2HCl | 313 | 63 |
| L-Glutamine | 146 | 584 |
| L-Histidine hydrochloride•H₂O | 210 | 42 |
| L-Isoleucine | 131 | 105 |
| L-Leucine | 131 | 105 |
| L-Lysine hydrochloride | 183 | 146 |
| L-Methionine | 149 | 30 |
| L-Phenylalanine | 165 | 66 |
| L-Serine | 105 | 42 |
| L-Threonine | 119 | 95 |
| L-Tryptophan | 204 | 16 |
| L-Tyrosine disodium. salt dihydrate | 261 | 104 |
| L-Valine | 117 | 94 |
| Choline chloride | 140 | 4 |
| D-Calcium pantothenate | 477 | 4 |
| Folic Acid | 441 | 4 |
| Niacinamide | 122 | 4 |
| Pyridoxine hydrochloride | 206 | 4 |
| Riboflavin | 376 | 0.4 |
| Thiamine hydrochloride | 337 | 4 |
| i-Inositol | 180 | 7.2 |
| Calcium Chloride (CaCl₂) (anhyd.) | 111 | 200 |
| Ferric Nitrate (Fe(NO₃)₃•9H₂O) | 404 | 0.1 |
| Magnesium Sulfate (MgSO₄) (anhyd.) | 120 | 97.67 |
| Potassium Chloride (KCl) | 75 | 400 |
| Sodium Bicarbonate (NaHCO₃) | 84 | 3700 |
| Sodium Chloride (NaCl) | 58 | 6400 |
| Sodium Phosphate monobasic | 138 | 125 |
| (NaH2PO4—H2O) | | |
| D-Glucose | 180 | 4500 |
| Fetal Calf Serum | | 0.05 mL/mL |
| | | mL/mL |
| Fetuin | | 0.05 |
| Epidermal Growth Factor (recombinant human) | | 10 ng/mL |
| Basic Fibroblast Growth Factor | | 1 ng/mL |
| (recombinant human) | | |
| Insulin (recombinant human) | | 0.01 |
| Dexamethasone | 392 | 0.4 μg/mL |
| | | μg/mL |

TABLE 2
Components of the differentiation medium for
porcine skeletal muscle satellite cells

| | Molecular | Content |
|---|---|---|
| Component name | weight | (mg/L) |
| Glycine | 75 | 30 |
| L-Arginine hydrochloride | 211 | 84 |
| L-Cystine•2HCl | 313 | 63 |
| L-Glutamine | 146 | 584 |
| L-Histidine hydrochloride•H₂O | 210 | 42 |
| L-Isoleucine | 131 | 105 |
| L-Leucine | 131 | 105 |
| L-Lysine hydrochloride | 183 | 146 |
| L-Methionine | 149 | 30 |
| L-Phenylalanine | 165 | 66 |
| L-Serine | 105 | 42 |
| L-Threonine | 119 | 95 |
| L-Tryptophan | 204 | 16 |
| L-Tyrosine disodium salt dihydrate | 261 | 104 |
| L-Valine | 117 | 94 |
| Choline chloride | 140 | 4 |
| D-Calcium pantothenate | 477 | 4 |
| Folic Acid | 441 | 4 |
| Niacinamide | 122 | 4 |
| Pyridoxine hydrochloride | 206 | 4 |
| Riboflavin | 376 | 0.4 |
| Thiamine hydrochloride | 337 | 4 |
| i-Inositol | 180 | 7.2 |
| Calcium Chloride (CaCl₂) (anhyd.) | 111 | 200 |
| Ferric Nitrate (Fe(NO₃)₃•9H₂O) | 404 | 0.1 |
| Magnesium Sulfate (MgSO₄) (anhyd.) | 120 | 97.67 |
| Potassium Chloride (KCl) | 75 | 400 |
| Sodium. Bicarbonate (NaHCO₃) | 84 | 3700 |
| Sodium Chloride (NaCl) | 58 | 6400 |
| Sodium Phosphate monobasic (NaH₂PO₄•H₂O) | 138 | 125 |
| D-Glucose | 180 | 4500 |
| Insulin | | 0.01 |

### Example 3

This example of the present disclosure provided a method for preparing block-shaped chicken-origin cultured meat, specifically as follows:

(1) Extraction of Chicken Skeletal Muscle Satellite Cells

A well-developed egg without damaged egg shell and obvious unremovable dirt was selected, gently wiped and disinfected with a 75% alcohol cotton ball. The air cell was gently broken by medical tweezers and the inner membrane attached to the shell was removed. The chicken embryo was taken out with elbow tweezers and dipped in 75% alcohol for 3 seconds. Subsequently, the disinfected chicken embryo was put into a Petri dish with PBS in advance, and rinsed with PBS twice. The PBS was absorbed. The chicken embryo thigh was cut off, and the outer fascia was torn off. Muscles were immobilized with a pair of tweezers. The muscles were torn and pulled with a pair of elbow tweezers tear, and the muscles attached to the bones on one thigh of the chicken embryo were torn into minced meat. The PBS was added to the minced chicken skeletal muscle tissue until it submerged the muscle tissue. The minced chicken skeletal muscle tissue was pipetted evenly and centrifuged at 800 rpm for 5 min, and the supernatant was discarded. The lower pellet was the target chicken skeletal muscle tissue explant. Subsequently, the above-mentioned tissue explant was resuspended in DMEM supplemented with 20% FBS, spread on a Petri dish, and placed in an incubator at 37° C. in 5% CO₂ for static culture. After culturing for one day, the skeletal muscle tissue explant was pipetted off the Petri dish, and fresh DMEM supplemented with 20% FBS was changed.

(2) Adherent and In Vitro Culture of Chicken Skeletal Muscle Satellite Cells

After the chicken skeletal muscle satellite cells extracted in step (1) covered more than 50% of the bottom area of the Petri dish, the culture medium was discarded, and the cells at the bottom of the Petri dish were rinsed once with DPBS. Then, the cells were digested with 1 mL of 0.25% pancreatin for 4.5 min at 37° C.; after that, 1 mL of 10% FBS in PBS was added to terminate the digestion, and the adherent cells were gently pipetted off, transferred to a centrifuge tube, and centrifuged at 900 rpm for 5 min; the supernatant was aspirated, and the lower pellets were primary chicken skeletal muscle satellite cells. Subsequently, the primary chicken skeletal muscle satellite cells were resuspended in 1 mL of DMEM supplemented with 20% FBS and counted. An appropriate quantity of the cells were inoculated in a cell factory (about 3×10⁶ cells/layer), and placed in an incubator at 37° C. in 5% CO₂ for static culture. The skeletal muscle satellite cells were passaged after expansion to cover 80% of the bottom area of the cell factory, and the number of skeletal muscle satellite cells was continuously expanded.

(3) 3D Bioprinting of Chicken Skeletal Muscle Satellite Cells

Under aseptic conditions, an appropriate quantity of the chicken skeletal muscle satellite cells obtained in step (2) were mixed with a volume of bioink GelMa, so that the volume ratio of the bioink was between 1% and 20%. The chicken skeletal muscle satellite cells were added into a bioprinter for 3D bioprinting into a block or grid in a sterile Petri dish.

(4) Proliferation Culture and Differentiation of Chicken Skeletal Muscle Satellite Cell Tissue

A chicken skeletal muscle satellite cell tissue printed in step (3) was placed in the special culture apparatus for a 3D biological tissue provided in Example 1 of the present disclosure, and the whole was cultured at 41° C. in 5% CO₂; after 1-2 days, when the cell morphology was stable, a proliferation medium for chicken skeletal muscle satellite cells (see Table 3 for specific components) was replaced with a differentiation medium for chicken skeletal muscle satellite cells (see Table 4 for specific components). After 3-5 days, the differentiation medium was replaced with the differentiation medium for further culture. Once the skeletal muscle satellite cells inside the 3D biological tissue differentiated and fused to form a unified whole, with the tissue elastic and the tissue surface lustrous, the 3D biological tissue culture and differentiation were completed, and a cultured meat structure unit was formed.

TABLE 3
Components of the proliferation medium for
chicken skeletal muscle satellite cells

| | Molecular | Content |
|---|---|---|
| Component name (in English) | weight | (mg/L) |
| Glycine | 75 | 30 |
| L-Arginine hydrochloride | 211 | 84 |
| L-Cystine•2HCl | 313 | 63 |
| L-Glutamine | 146 | 584 |
| L-Histidine hydrochloride•H₂O | 210 | 42 |
| L-Isoleucine | 131 | 105 |
| L-Leucine | 131 | 105 |
| L-Lysine hydrochloride | 183 | 146 |
| L-Methionine | 149 | 30 |
| L-Phenylalanine | 165 | 66 |
| L-Serine | 105 | 42 |
| L-Threonine | 119 | 95 |
| L-Tryptophan | 204 | 16 |
| L-Tyrosine disodium salt dihydrate | 261 | 104 |
| L-Valine | 117 | 94 |
| Choline chloride | 140 | 4 |
| D-Calcium pantothenate | 477 | 4 |
| Folic Acid | 441 | 4 |
| Niacinamide | 122 | 4 |
| Pyridoxine hydrochloride | 206 | 4 |
| Riboflavin. | 376 | 0.4 |
| Thiamine hydrochloride | 337 | 4 |
| i-Inositol | 180 | 7.2 |
| Calcium Chloride (CaCl₂) (anhyd.) | 111 | 200 |
| Feme Nitrate (Fe(NO₃)₃•9H₂O) | 404 | 0.1 |
| Magnesium Sulfate (MgSO₄) (anhyd.) | 120 | 97.67 |
| Potassium Chloride (KCl) | 75 | 400 |
| Sodium Bicarbonate (NaHCO₃) | 84 | 3700 |
| Sodium Chloride (NaCl) | 58 | 6400 |
| Sodium Phosphate monobasic | 138 | 125 |
| D-Glucose | 180 | 4500 |
| Fetal Calf Serum | | 0.2 mL/mL |

TABLE 4
Components of the differentiation medium for
chicken skeletal muscle satellite cells

| | Molecular | Content |
|---|---|---|
| Component name (in English) | weight | (mg/L) |
| Glycine | 75 | 30 |
| L-Arginine hydrochloride | 211 | 84 |
| L-Cystine•2HCl | 313 | 63 |
| L-Glutamine | 146 | 584 |
| L-Histidine hydrochloride•H₂O | 210 | 42 |
| L-Isoleucine | 131 | 105 |
| L-Leucine | 131 | 105 |
| L-Lysine hydrochloride | 183 | 146 |
| L-Methionine | 149 | 30 |
| L-Phenylalanine | 165 | 66 |
| L-Serine | 105 | 42 |
| L-Threonine | 119 | 95 |
| L-Tryptophan | 204 | 16 |
| L-Tyrosine disodium salt dihydrate | 261 | 104 |
| L-Valine | 117 | 94 |
| Choline chloride | 140 | 4 |
| D-Calcium pantothenate | 477 | 4 |
| Folic Acid | 441 | 4 |
| Niacinamide | 122 | 4 |
| Pyridoxine hydrochloride | 206 | 4 |
| Riboflavin | 376 | 0.4 |
| Thiamine hydrochloride | 337 | 4 |
| i-Inositol | 180 | 7.2 |
| Calcium Chloride (CaCl₂) (anhyd.) | 111 | 200 |
| Ferric Nitrate (Fe(NO₃)₃•9H₂O) | 404 | 0.1 |
| Magnesium Sulfate (MgSO₄) (anhyd.) | 120 | 97.67 |
| Potassium Chloride (KCl) | 75 | 400 |
| Sodium Bicarbonate (NaHCO₃) | 84 | 3700 |
| Sodium Chloride (NaCl) | 58 | 6400 |
| Sodium Phosphate monobasic (NaH₂PO₄•H₂O) | 138 | 125 |
| D-Glucose | 180 | 4500 |

### Example 4

This example provided a method for preparing block-shaped porcine-derived cultured meat the same as Example 2 with exception that the adherent culture method was replaced with the suspension culture method by means of loading on the surface of a microcarrier microsphere during the in vitro culture of porcine skeletal muscle satellite cells in step (2).

### Example 5

This example provided a method for preparing block-shaped chicken-derived cultured meat the same as Example 3 with exception that the adherent culture method was replaced with the suspension culture method by means of loading on the surface of a microcarrier microsphere during the in vitro culture of chicken skeletal muscle satellite cells in step (2).

### Comparative Example 1

This comparative example provided a method for preparing porcine-derived cultured meat the same as Example 2 with exception that the tissue explant adherent method was replaced with the digestion tissue explant adherent method when extracting skeletal muscle satellite cells in step (1).

### Comparative Example 2

This comparative example provided a method for preparing porcine-derived cultured meat the same as Example 1 with exception that static culture in an incubator was used, instead of the special culture apparatus for a 3D biological tissue provided in Example 1, during the proliferation culture and differentiation of the 3D porcine skeletal muscle satellite cell tissue in step (4).

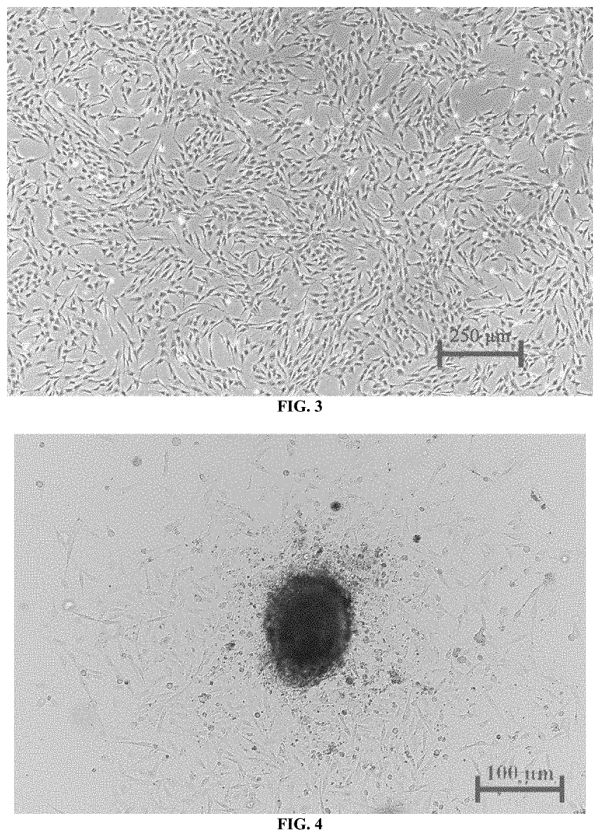

FIGS. 2 and 3 illustrate the morphology of porcine skeletal muscle satellite cells cultured for 48 h and 72 h in step (2) of Example 2 of the present disclosure, respectively. As can be seen from the figures, the primary porcine skeletal muscle satellite cells were fusiform. With the extension of the culture time, tentacles gradually increased, showing a satellite radial cell morphology, consistent shape, and rapid proliferation.

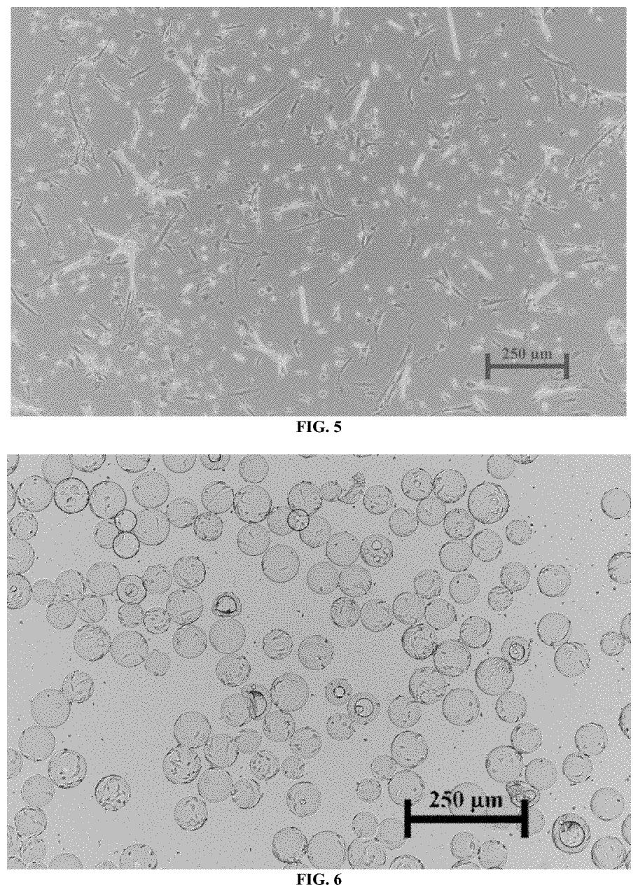

FIGS. 4 and 5 illustrate the morphology of chicken skeletal muscle satellite cells cultured for 24 h and 48 h in step (2) of Example 3 of the present disclosure, respectively. As can be seen from the figures, the extraction efficiency of primary chicken skeletal muscle satellite cells was high (approximately 5×10⁵ primary chicken skeletal muscle satellite cells were obtained per gram of chicken skeletal muscle satellite cell tissue), the cell growth rate was extremely fast, and the morphology was stable.

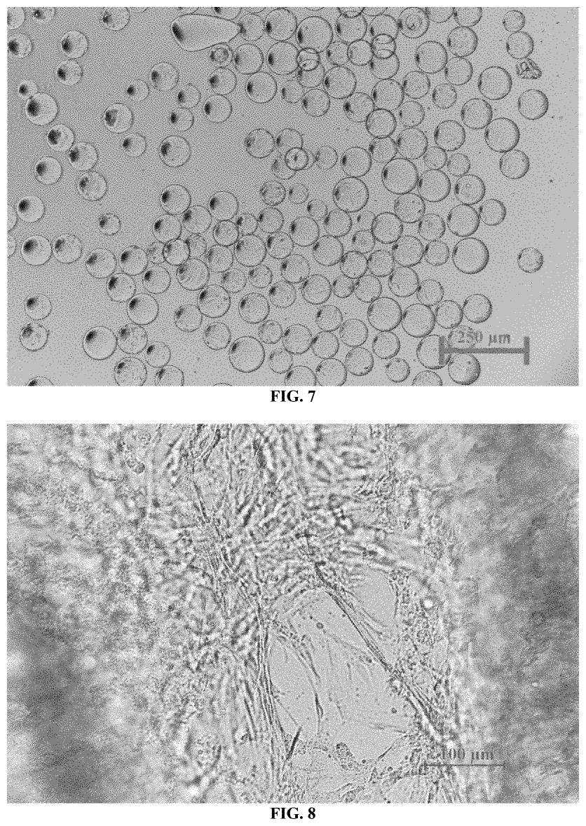

FIG. 6 illustrates the morphology of porcine skeletal muscle satellite cells cultured for 72 h in step (2) of Example 4 of the present disclosure; FIG. 7 illustrates the morphology of chicken skeletal muscle satellite cells cultured for 48 h in step (2) of Example 5 of the present disclosure. According to the comparison of FIGS. 6 and 7 versus FIGS. 3 and 5, during the in vitro culture, both the adherent culture method and the suspension culture method by means of loading on the surface of a microcarrier microsphere were feasible. However, the suspension culture method by means of loading on the surface of a microcarrier microsphere led to a higher cell expansion density. According to calculations, if a microcarrier microsphere are used as carriers for the growth of skeletal muscle satellite cells (MSCs) in suspension culture spinner flasks for suspension culture, 3.5×10⁷ chicken MSCs or 2.5×10⁷ porcine MSCs can be produced per milliliter of culture system, which can increase the use efficiency of the culture medium by 20-50 times; moreover, the spinner flasks were repeatable, which may effectively reduce the production cost.

FIG. 8 illustrates the morphology of porcine skeletal muscle satellite cells cultured for 5 days in step (2) of Example 2 of the present disclosure. As can be seen from the figure, the skeletal muscle satellite cells in the 3D culture state proliferated rapidly and grew substantial tentaculiform skeletons, and the mutual contact and fusion occurred, indicating that the cells grew well in the bioink matrix.

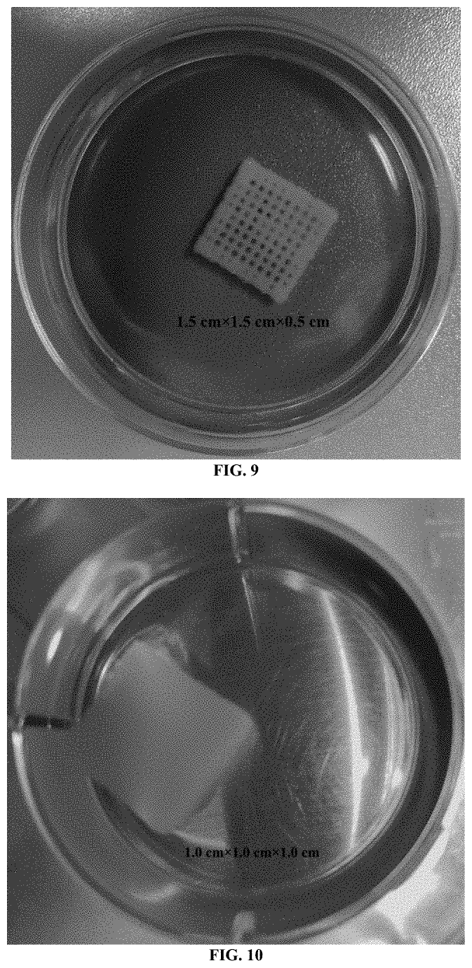

FIGS. 9 and 10 illustrate the morphology of a 3D porcine skeletal muscle satellite cell tissue cultured for 10 and 15 days in Example 2 of the present disclosure, respectively. It can be seen that the block-shaped cultured meat had been substantially formed.

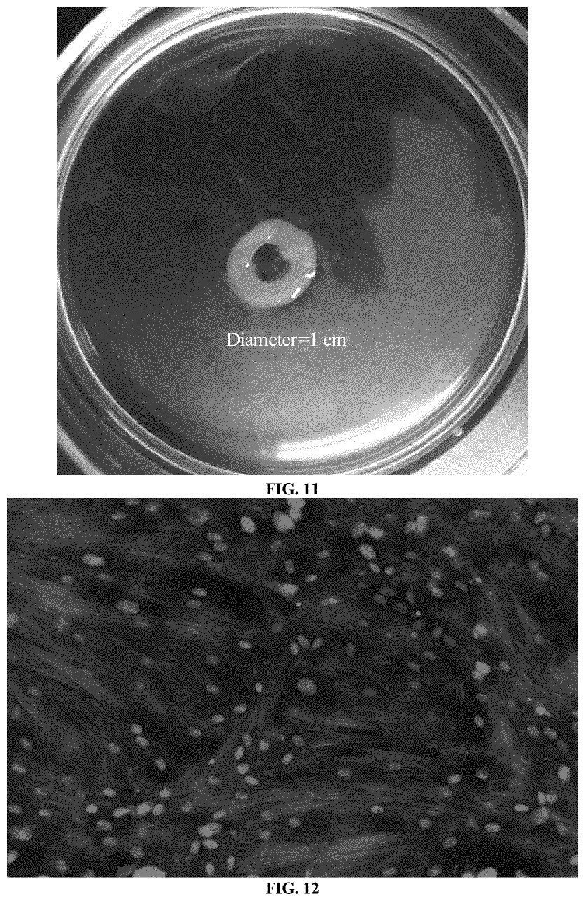

FIG. 11 illustrates the morphology of a 3D chicken skeletal muscle satellite cell tissue cultured for 15 days in Example 3 of the present disclosure. It can be seen that the block-shaped cultured meat with a diameter of 1 cm had been formed.

FIG. 12 illustrates the myogenesis of porcine muscle stem cells in Example 2 of the present disclosure; FIG. 13 illustrates the myogenesis of chicken muscle stem cells in Example 3 of the present disclosure. As can be seen from the figures, the cells were in a fully extended state and differentiate into 3D fibrous myotubes in situ, and the cell proliferation and differentiation viability still existed after 3D forming.

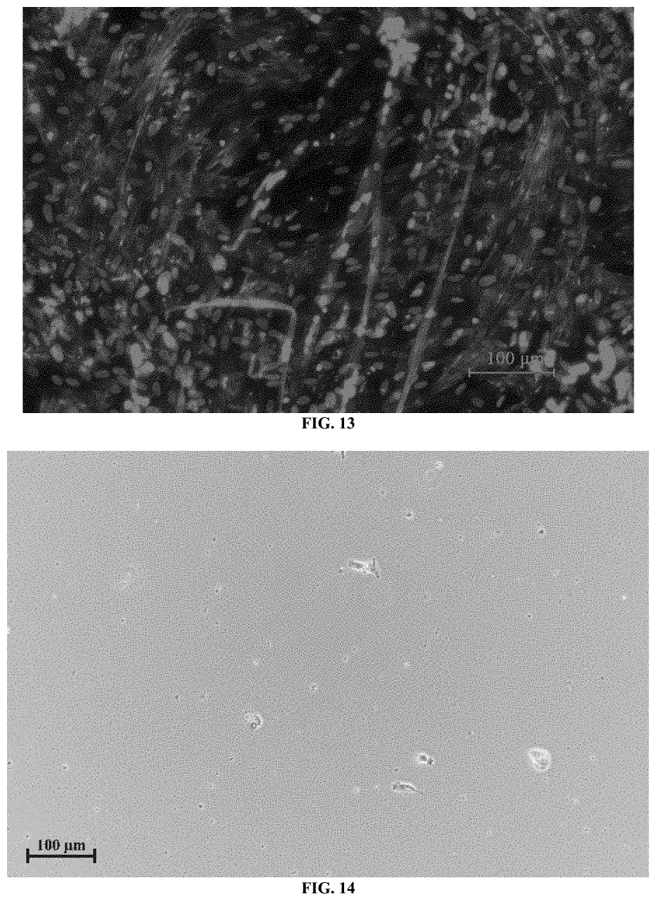

FIG. 14 illustrates the morphology of porcine skeletal muscle satellite cells extracted by the digestion tissue explant adherent method in Comparative Example 1 of the present disclosure; FIG. 15 illustrates the morphology of porcine skeletal muscle satellite cells extracted by the tissue explant adherent method in Example 2 of the present disclosure. As can be seen from the figures, extraction by the tissue explant adherent method reduced the damage to skeletal muscle satellite cells, and the animal skeletal muscle satellite cells obtained exhibited high purity, uniform and stable morphology, stable stemness maintenance, high regenerative activity, and fast expansion rate.

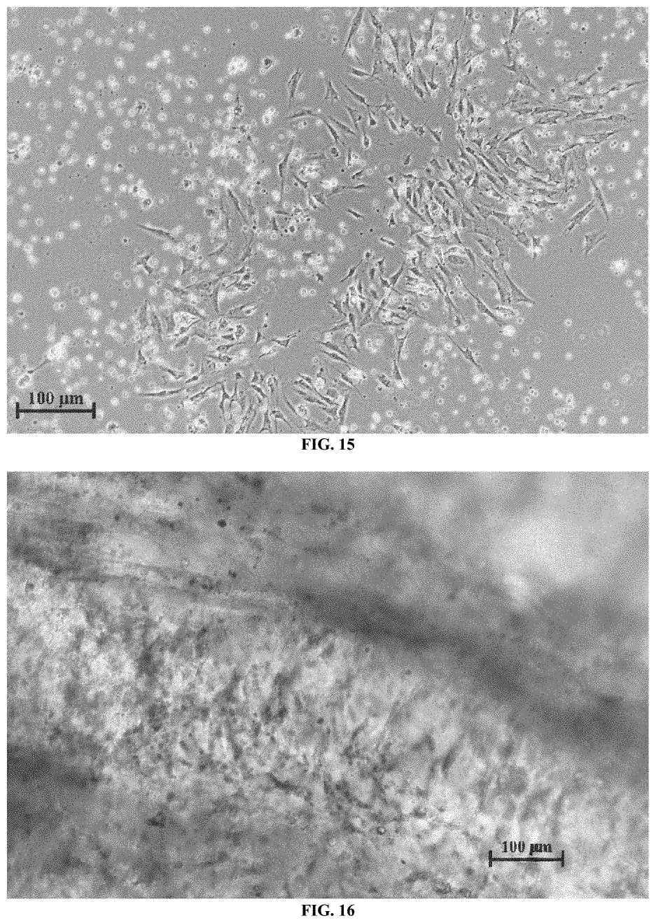

FIG. 16 illustrates the morphology of porcine skeletal muscle satellite cells after static culture in Comparative Example 2 of the present disclosure. As can be seen from the figure, substantial cell deaths occurred in the middle of the tissue explant, which can be in sharp contrast with FIG. 8.

The results show that the examples of the present disclosure provide an effective method for preparing block-shaped cultured meat. The method for extracting animal skeletal muscle satellite cells provided in the examples of the present disclosure provides seed cells for cultured meat production with high efficiency. The in vitro expansion method of animal skeletal muscle satellite cells provided in the examples of the present disclosure provides substantial animal skeletal muscle satellite cells for the production of the cultured meat in a relatively short period of time at low cost with simple operations. The 3D forming method of animal skeletal muscle satellite cells provided in the examples of the present disclosure realizes rapid and automatic production. The method of proliferation culture and differentiation of the cell tissue provided in the examples of the present disclosure realizes the overall culture and differentiation of the cultured meat, which is beneficial to enhance the food properties of the cultured meat and improve the eating taste of the cultured meat.

Finally, it should be noted that the above examples are only intended to illustrate, but not to limit, the technical solutions of the present disclosure; although the present disclosure has been described in detail with reference to the foregoing examples, those of ordinary skill in the art should understand that: the technical solutions recorded in the foregoing examples may be still modified, or some of the technical features may be equivalently substituted; these modifications or substitutions do not cause the essence of the corresponding technical solutions to depart from the spirit and scope of the technical solutions of the examples of the present disclosure.

### INDUSTRIAL APPLICABILITY

The present disclosure provides a special culture apparatus for a 3D biological tissue, and a method for preparing block-shaped cultured meat. The special culture apparatus for a 3D biological tissue includes a 3D biological tissue culture tank for accommodating a 3D biological tissue, and a liquid storage tank for containing a culture medium; the 3D biological tissue culture tank is connected to the liquid storage tank by means of a pipeline to form a circuit for the culture medium to circularly flow; and an opening of the 3D biological tissue culture tank is provided with a sealing plug, an inner side of the sealing plug is provided with a plurality of culture medium infusion needles that penetrate the 3D biological tissue when in use. The special culture apparatus for a 3D biological tissue provided by the present disclosure may effectively guarantee the uniform growth and differentiation of cells in the 3D biological tissue and is suitable to prepare the block-shaped cultured meat. The method for preparing block-shaped cultured meat provided by the present disclosure may significantly increase the preparation scale of cultured animal meat, and truly achieve the preparation of high-quality block-shaped cultured meat with a chewing sensation of meat products, which has excellent economic value and application prospect.

1. A special culture apparatus for a 3D biological tissue, comprising:
a 3D biological tissue culture tank for accommodating a 3D biological tissue, and
a liquid storage tank for containing a culture medium; wherein
the 3D biological tissue culture tank is connected to the liquid storage tank by means of a pipeline to form a circuit for the culture medium to circularly flow; and
an opening of the 3D biological tissue culture tank is provided with a sealing plug, with an inner side of the sealing plug provided with a plurality of culture medium infusion needles that penetrate the 3D biological tissue when in use.

2. A method for preparing block-shaped cultured meat, comprising steps of:
mixing animal skeletal muscle satellite cells with bioink for 3D bioprinting, and placing a printed 3D animal skeletal muscle satellite cell tissue in the special culture apparatus for a 3D biological tissue according to claim 1 for proliferation culture and differentiation.

3. The method for preparing block-shaped cultured meat according to claim 2, wherein the bioink is methacrylic anhydride-modified gelatin (GelMa) and/or nanocellulose.

4. The method for preparing block-shaped cultured meat according to claim 2, wherein when the animal skeletal muscle satellite cells are porcine skeletal muscle satellite cells, the printed 3D animal skeletal muscle satellite cell tissue is placed in the special culture apparatus for a 3D biological tissue according to claim 1, and the whole is cultured at 36.5-37.5° C. in 5% CO₂; after 1-2 days, when cell morphology is stable, a proliferation medium is replaced with a differentiation medium; after 3-5 days, the differentiation medium is replaced with the proliferation medium; and further culture is continued until the cells differentiate and fuse to form the block-shaped cultured meat.

5. The method for preparing block-shaped cultured meat according to claim 4, wherein when the animal skeletal muscle satellite cells are porcine skeletal muscle satellite cells, the proliferation medium used comprises 8-12 ng/mL epidermal growth factor, 0.5-2 ng/mL fibroblast growth factor, 0.005-0.015 mg/L insulin, and 0.3-0.5 μg/mL dexamethasone, and the differentiation medium used comprises 0.005-0.015 mg/L insulin.

6. The method for preparing block-shaped cultured meat according to claim 2, wherein when the animal skeletal muscle satellite cells are chicken skeletal muscle satellite cells, the printed 3D animal skeletal muscle satellite cell tissue is placed in the special culture apparatus for a 3D biological tissue according to claim 1, and the whole is cultured at 40.5-41.5° C. in 5% CO₂; after 1-2 days, when cell morphology is stable, a proliferation medium is replaced with a differentiation medium; after 3-5 days, the differentiation medium is replaced with the proliferation medium; and further culture is continued until the cells differentiate and fuse to form the block-shaped cultured meat.

7. The method for preparing block-shaped cultured meat according to claim 6, wherein when the animal skeletal muscle satellite cells are chicken skeletal muscle satellite cells, the proliferation medium used is a McCoy's 5A Medium supplemented with 10-20% chicken serum or fetal bovine serum (FBS), and the differentiation medium used is a McCoy's 5A Medium supplemented with 0-5% chicken serum or FBS.

8. The method for preparing block-shaped cultured meat according to claim 2, wherein the animal skeletal muscle satellite cells are obtained by extraction and in vitro culture, wherein the porcine skeletal muscle satellite cells are extracted from a skeletal muscle tissue of a neonatal animal, and the chicken skeletal muscle satellite cells are extracted from an embryo in an incubator.

9. The method for preparing block-shaped cultured meat according to claim 8, wherein the in vitro culture is implemented by an adherent culture method or a suspension culture method by means of loading on the surface of a microcarrier microsphere.

10. Block-shaped cultured meat, wherein the block-shaped cultured meat is prepared by the method for preparing block-shaped cultured meat according to claim 2.

11. The block-shaped cultured meat according to claim 10, wherein the bioink is methacrylic anhydride-modified gelatin (GelMa) and/or nanocellulose.

12. The block-shaped cultured meat according to claim 10, wherein when the animal skeletal muscle satellite cells are porcine skeletal muscle satellite cells, the printed 3D animal skeletal muscle satellite cell tissue is placed in the special culture apparatus for a 3D biological tissue according to claim 1, and the whole is cultured at 36.5-37.5° C. in 5% CO₂; after 1-2 days, when cell morphology is stable, a proliferation medium is replaced with a differentiation medium; after 3-5 days, the differentiation medium is replaced with the proliferation medium; and further culture is continued until the cells differentiate and fuse to form the block-shaped cultured meat.

13. The block-shaped cultured meat according to claim 10, wherein when the animal skeletal muscle satellite cells are porcine skeletal muscle satellite cells, the proliferation medium used comprises 8-12 ng/mL epidermal growth factor, 0.5-2 ng/mL fibroblast growth factor, 0.005-0.015 mg/L insulin, and 0.3-0.5 μg/mL dexamethasone, and the differentiation medium used comprises 0.005-0.015 mg/L insulin.

14. The block-shaped cultured meat according to claim 10, wherein when the animal skeletal muscle satellite cells are chicken skeletal muscle satellite cells, the printed 3D animal skeletal muscle satellite cell tissue is placed in the special culture apparatus for a 3D biological tissue according to claim 1, and the whole is cultured at 40.5-41.5° C. in 5% CO₂; after 1-2 days, when cell morphology is stable, a proliferation medium is replaced with a differentiation medium; after 3-5 days, the differentiation medium is replaced with the proliferation medium; and further culture is continued until the cells differentiate and fuse to form the block-shaped cultured meat.

15. The block-shaped cultured meat according to claim 10, wherein when the animal skeletal muscle satellite cells are chicken skeletal muscle satellite cells, the proliferation medium used is a McCoy's 5A Medium supplemented with 10-20% chicken serum or fetal calf serum (FCS), and the differentiation medium used is a McCoy's 5A Medium supplemented with 0-5% chicken serum or FCS.

16. The block-shaped cultured meat according to claim 10, wherein the animal skeletal muscle satellite cells are obtained by extraction and in vitro culture, wherein the porcine skeletal muscle satellite cells are extracted from a skeletal muscle tissue of a neonatal animal, and the chicken skeletal muscle satellite cells are extracted from an embryo in an incubator.

17. The block-shaped cultured meat according to claim 10, wherein the in vitro culture is implemented by an adherent culture method or a suspension culture method by means of loading on the surface of a microcarrier microsphere.
